(19) United States
(12) Patent Application Publication
(10) Pub. No.: US 2025/0376651 A1
(43) Pub. Date: Dec. 11, 2025

(54) Culture Apparatus and Printing Method

(71) Applicant: SHIMADZU CORPORATION, Kyoto-shi (JP)

(72) Inventor: Yamato MAEDA, Kyoto-shi (JP)

(21) Appl. No.: 19/230,020

(22) Filed: Jun. 5, 2025

(30) Foreign Application Priority Data
Jun. 5, 2024 (JP) 2024-091184

(51) Int. Cl. C12M 3/00; B33Y 10/00; B33Y 30/00; C12M 1/00; C12M 1/36

(52) U.S. Cl. CPC C12M 21/08; B33Y 10/00; B33Y 30/00; C12M 29/00; C12M 41/48

(57) ABSTRACT

A culture apparatus includes a bioprinter that linearly prints bioink, a housing configured to accommodate a substrate that supports the bioink, a driver that moves the bioprinter and the housing relatively to each other, and a controller. The controller is configured to control the driver to locate an outlet of the bioprinter at a printing start position in the housing and thereafter to move the outlet in a first direction in the substrate within the housing. The controller is configured to control a pump for the bioink to start application of a pressure to the bioink after the outlet is inserted in the substrate and before the outlet starts moving from the printing start position in the first direction.

### CROSS REFERENCE TO RELATED APPLICATIONS

This nonprovisional application is based on Japanese Patent Application No. 2024-091184 filed with the Japan Patent Office on Jun. 5, 2024, the entire contents of which are hereby incorporated by reference.

### BACKGROUND OF THE INVENTION

### Field of the Invention

The present disclosure relates to a culture apparatus for bioprinting for linearly printing bioink containing cells to obtain a linear cell tissue, and a method of printing bioink in a substrate that supports the bioink, the bioink containing cells to be a cell tissue by being cultured.

### Description of the Background Art

A technique to construct a cell tissue by bioprinting has been studied. Studies to construct a cell tissue by bioprinting and to use the obtained cell tissue as steak meat or processed meat as food have progressed.

WO2021/193980 discloses linear printing of bioink in a printing bath composed of a supporting bath material and a collagen layer.

### SUMMARY OF THE INVENTION

In linearly printing bioink, a syringe is inserted in a substrate that three-dimensionally supports cells, and bioink is discharged while an operation to draw up the syringe is performed after an outlet of the syringe reaches a printing start position. When the operation to draw up the syringe is started simultaneously with start of an operation to discharge bioink, for such a reason as entry of the substrate in the syringe at the time of insertion of the syringe, printing of bioink may not be started from the printing start position. A printed line of the bioink may not be as long as a desired length and a cell tissue having a desired length may not be obtained.

One object of the present disclosure is to provide a technique to enable printing of bioink by a desired length in order to obtain a linear cell tissue having a desired length.

A culture apparatus in the present disclosure is a culture apparatus for bioprinting for linearly printing bioink containing cells to obtain a linear cell tissue. The culture apparatus includes a bioprinter that discharges the bioink and linearly prints the bioink, a housing configured to accommodate a substrate that supports the bioink, a driver that moves the bioprinter and the housing relatively to each other, and a controller. The bioprinter includes a tube where the bioink is accommodated and a pump configured to apply a pressure to the bioink in a direction toward an outlet of the tube. The controller is configured to control the driver to locate the outlet at a printing start position within the housing accommodating the substrate and thereafter to move the outlet in a first direction in the substrate within the housing. The controller is configured to control the pump to start application of a pressure to the bioink in a direction toward the outlet after the outlet is inserted in the substrate and before the outlet starts moving from the printing start position in the first direction.

A printing method in the present disclosure is a method of printing bioink in a substrate that supports the bioink, the bioink containing cells to be a cell tissue by being cultured. The printing method includes locating an outlet of a bioprinter at a printing start position in the substrate, the bioprinter being configured to linearly print the bioink, moving within the substrate, the outlet from the printing start position in a first direction, and starting application of a pressure to the bioink in the bioprinter in a direction toward the outlet after the outlet is inserted in the substrate and before the outlet starts moving in the first direction.

The foregoing and other objects, features, aspects, and advantages of this invention will become more apparent from the following detailed description of this invention when taken in conjunction with the accompanying drawings.

### BRIEF DESCRIPTION OF THE DRAWINGS

FIG. 1 is a diagram schematically showing a structure of a culture apparatus according to one embodiment.

FIG. 2 is a schematic cross-sectional view of a bioprinter.

FIG. 3 is a diagram showing overview of a printing operation.

FIG. 4 is a schematic diagram showing an example of successful printing.

FIG. 5 is a schematic diagram showing an example of failed printing.

FIG. 6 is a flowchart showing a process to be performed by a controller.

### DESCRIPTION OF THE PREFERRED EMBODIMENTS

An embodiment of the present disclosure will be described in detail below with reference to the drawings. The same or corresponding elements in the drawings have the same reference characters allotted and description thereof will not be repeated.

### [Culture Apparatus]

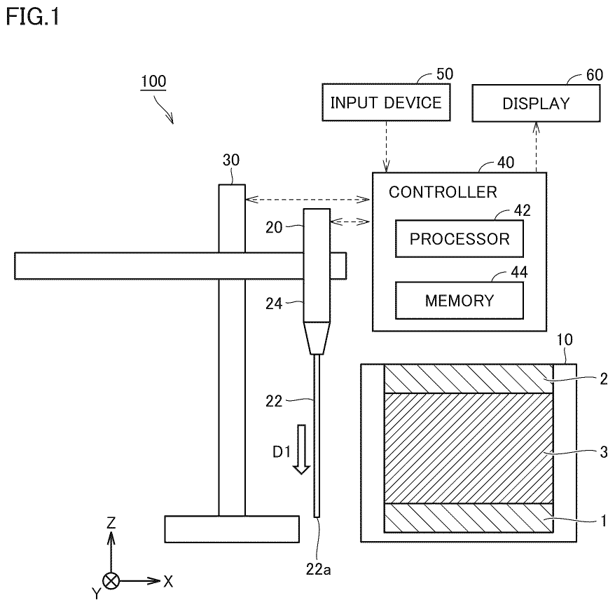

FIG. 1 is a diagram schematically showing a structure of a culture apparatus according to one embodiment. A culture apparatus 100 is a culture apparatus for bioprinting for linearly printing bioink containing cells to obtain a linear cell tissue. Cells are not particularly limited so long as they form a fibrous cell tissue by being linearly printed and cultured. Cells may be, for example, cells derived from animals, and may be cells derived from human or cells derived from an animal other than human. Cells employed in the present embodiment are, for example, skeletal muscle cells. Skeletal muscle cells may be cells derived from muscles or cells derived from stem cells.

Culture apparatus 100 includes a housing 10, a bioprinter 20, a driver 30, a controller 40, an input device 50, and a display 60. Controller 40 controls bioprinter and driver 30. Controller 40 receives input from input device 50 and has display 60 show an image for receiving input from input device 50.

Housing 10 is configured to accommodate a substrate that three-dimensionally supports bioink. The substrate includes a first supporter 1, a second supporter 2, and a support material 3. First supporter 1, support material 3, and second supporter 2 are accommodated in this order from a bottom surface side of housing 10. In FIG. 1, a direction from first supporter 1 toward second supporter 2 is defined as a Z-axis direction and a plane perpendicular to the Z-axis direction is defined as an XY plane.

Culture apparatus 100 may be provided, with a substrate being arranged in advance in housing 10, or may be configured such that a substrate can subsequently be inserted and arranged in housing 10. In housing 10, areas where first supporter 1, second supporter 2, and support material 3 are arranged may be separated from one another by a wall or the like. In this case, the wall that separates the areas may be provided with an opening through which a tube 22 of bioprinter 20 can be inserted.

When bioink is linearly printed, first supporter 1 and second supporter 2 each support an end of the linear bioink. First supporter 1 and second supporter 2 should only support cells contained in the bioink by binding therewith, and each of them is composed of a material selected as appropriate by a person skilled in the art. First supporter 1 and second supporter 2 may be in any of a solid state and a liquid state. The solid state is assumed to encompass a gel state. A physical property of first supporter 1 and second supporter 2 is not particularly limited, and first supporter 1 and second supporter 2 may have, for example, such a physical property as varying in shape under a prescribed condition or not varying in shape. The prescribed condition includes, for example, a temperature, a pressure, an electrical stimulus, pH, and the like. First supporter 1 and second supporter 2 are, for example, collagen, collagen nanofibers, or the like. A type of collagen is not particularly limited. For example, first supporter 1 and second supporter 2 may be composed of a plurality of kinds of collagens different in type. First supporter 1 and second supporter 2 may be different from each other in composition. In the present embodiment, first supporter 1 and second supporter 2 will be described as being assumed as collagen nanofibers.

Support material 3 is composed of a soluble material. For example, support material 3 is a solution obtained by dissolving a high polymeric substance such as gelatin, agar, or gellan gum in an aqueous solvent. Support material 3 may be in a gel state or a sol state. Support material 3 may have such a thixotropic property that it becomes lower in viscosity and liquefied when force is applied thereto and gradually recovers its viscosity as the force is removed. support material 3 may be, for example, a mixed solution prepared by crushing a gelated sample and dispersing the sample in a solvent such as a liquid culture medium. The gelated sample is prepared, for example, by dissolving a high polymeric substance such as gelatin, agar, or gellan gum in an aqueous solvent and gelating the same. Support material 3 will be described as being assumed as the mixed solution obtained by crushing a sample obtained by gelation of a gelatin solution and dispersing the sample in a liquid culture medium.

Bioprinter 20 includes tube 22 where bioink is accommodated and a pump 24. Pump 24 is configured to apply a pressure to bioink accommodated in tube 22 in a direction toward an outlet 22a of tube 22 (which will also be referred to as a “direction of discharge D1” below). Pump 24 is, for example, a plunger pump, and it suctions a sample (bioink) into tube 22 or discharges the sample from tube 22 by causing a piston to make reciprocating motion as will be described later.

Driver 30 moves housing 10 and bioprinter 20 relatively to each other. In the present embodiment, driver 30 is configured to move bioprinter 20. Driver 30 moves bioprinter 20, for example, horizontally and upward and downward. Driver 30 can freely move bioprinter 20, for example, with a solenoid actuator or a stepping motor.

Controller 40 is a device for control of culture apparatus 100 as a whole, and includes a processor 42 and a memory 44. Processor 42 is, for example, a central processing unit (CPU), and it is computing circuitry that performs prescribed computing processing described in a program. Memory 44 includes a non-volatile memory or a volatile memory such as a read only memory (ROM) or a random access memory (RAM) and/or a large-capacity storage such as a hard disc drive (HDD) or a solid state drive (SSD). Processor 42 reads a program and data stored in memory 44 and controls pump 24, driver 30, and the like included in culture apparatus 100.

### [Configuration of Bioprinter]

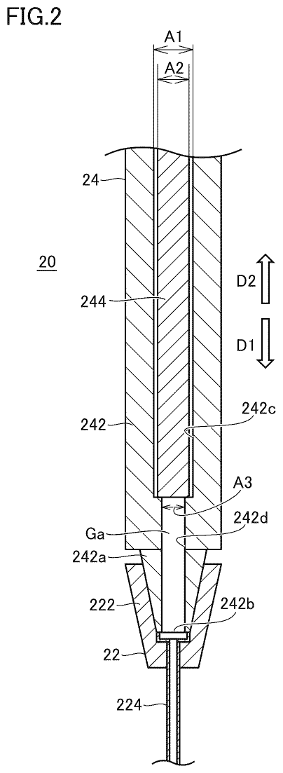

FIG. 2 is a schematic cross-sectional view of the bioprinter. Tube 22 is attachable to and removable from pump 24 and includes a connection port 222 and a nozzle 224. Tube 22 can be attached to pump 24 by attaching connection port 222 to a connection port 242a of a pump housing 242 of pump 24.

Pump 24 includes pump housing 242 and a piston 244 configured to make reciprocating motion in pump housing 242. Pump housing 242 is configured to be provided with a gap Ga of a prescribed volume between an opening end 242b of connection port 242a and piston 244 when piston 244 moves to a position closest to connection port 242a in reciprocating motion.

The prescribed volume should only be equal to or larger than a volume of bioink to be accommodated in tube 22 in one printing operation. In an example shown in FIG. 2, in pump housing 242, an accommodation portion 242c where piston 244 is accommodated and a tip end 242d that communicates with opening end 242b are formed. An inner diameter A1 of accommodation portion 242c is larger than an outer diameter A2 of piston 244. An inner diameter A3 of tip end 242d at a portion of boundary of accommodation portion 242c is smaller than outer diameter A2 of piston 244. Therefore, when piston 244 moves to the position closest to connection port 242a in reciprocating motion, gap Ga corresponding to the volume of tip end 242d is provided in pump housing 242.

Since tube 22 is attachable to and removable from pump 24 in the present embodiment, it is readily replaceable. Therefore, tube 22 is disposable and pump 24 is reusable, which is hygienic and economical. Since gap Ga is provided in pump housing 242, entry of bioink in pump housing 242 at the time of suction of bioink can be prevented. Therefore, in repeated use of bioprinter 20, time and efforts for cleaning and sterilization of the inside of pump 24 can be saved.

A direction opposite to direction of discharge D1 will be referred to as a direction of suction D2 below. Pump 24 suctions bioink into nozzle 224 by movement of piston 244 in direction of suction D2 while outlet 22a of nozzle 224 is located in bioink. Alternatively, pump 24 discharges bioink from outlet 22a by movement of piston 244 in direction of discharge D1 while bioink is accommodated in nozzle 224.

### [Overview of Printing Operation]

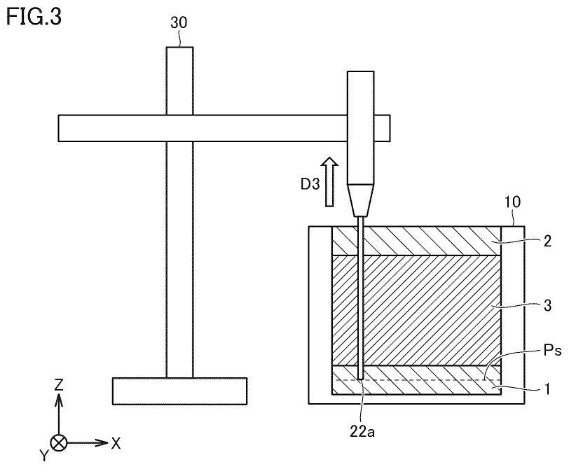

A series of operations in printing bioink in the substrate will be described with reference to FIG. 3. FIG. 3 is a diagram showing overview of a printing operation.

Controller 40 controls driver 30 such that outlet 22a of bioprinter 20 is located at a printing start position Ps in housing 10 while bioink is accommodated in tube 22. Specifically, controller 40 controls pump 24 and driver 30 to have a predetermined volume of bioink suctioned into tube 22. Thereafter, controller 40 controls driver 30 to move tube 22 of bioprinter 20 from an upper portion of housing 10 toward a bottom surface (a negative direction along a Z axis in the figure) of housing 10 until outlet 22a is located at printing start position Ps. Printing start position Ps is located in first supporter 1.

Controller 40 has outlet 22a located at printing start position Ps, and thereafter controls driver 30 to move outlet 22a in a direction of draw-up D3. Direction of draw-up D3 is a direction from first supporter 1 toward second supporter 2 and a positive direction along the Z axis in the figure.

Controller 40 controls bioprinter 20 and driver 30 such that bioink is discharged from outlet 22a while outlet 22a moves in direction of draw-up D3. Bioink is thus linearly printed in the substrate.

Controller 40 controls pump 24 to start application of a pressure to bioink in tube 22 in direction of discharge D1 after outlet 22a is inserted in the substrate and before outlet 22a starts moving from printing start position Ps in direction of draw-up D3.

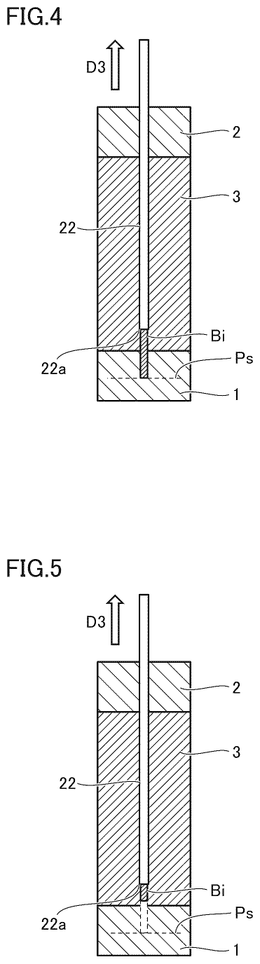

FIG. 4 is a schematic diagram showing an example of successful printing. FIG. is a schematic diagram showing an example of failed printing. For example, as shown in FIG. 5, when application of the pressure to bioink in direction of discharge D1 is started at the timing of start of movement of outlet 22a from printing start position Ps in direction of draw-up D3, for such a reason as entry of the substrate (first supporter 1, second supporter 2, or support material 3) in the syringe at the time of insertion of tube 22, printing of bioink Bi may not be started from printing start position Ps.

In particular, in bioprinter 20 according to the present embodiment, gap Ga is provided in pump housing 242. Since a compression ratio of air is higher than a compression ratio of the substrate, bioprinter 20 is in such a configuration that, under the influence by gap Ga, air in gap Ga is compressed at the time of insertion of nozzle 224 in the substrate and the substrate is more likely to enter nozzle 224. Since the compression ratio of air is higher than a compression ratio of bioink, also at the time of discharge of bioink, air in gap Ga is compressed at the timing of start of movement of piston 244 in direction of discharge D1 and thereafter the pressure is transmitted to bioink in nozzle 224. Therefore, there is a time lag between the timing of start of application of the pressure to bioink in direction of discharge D1 and the timing of actual discharge of bioink from outlet 22a. When application of the pressure is started at the timing of start of movement in direction of draw-up D3, this time lag causes such a disadvantage that printing is not started from desired printing start position Ps. Consequently, the printed line of bioink may not be as long as a desired length and a cell tissue having a desired length may not be obtained.

When printing is not started from desired printing start position Ps but started from within support material 3 not within first supporter 1, one end of bioink cannot be supported by first supporter 1 and a desired growth environment cannot be provided to skeletal muscle cells which are cells in bioink.

In the present embodiment, application of the pressure to bioink in direction of discharge D1 is started before start of movement of outlet 22a from printing start position Ps in direction of draw-up D3, so that printing is started from desired printing start position Ps as shown in FIG. 4 and hence bioink can be printed by a desired length.

Since printing is started from printing start position Ps, one end of printed linear bioink can be supported by first supporter 1.

Controller 40 should only set timing of start of application of the pressure in direction of discharge D1 to come before start of movement in direction of draw-up D3, and the timing may come, for example, during movement of outlet 22a within housing in the direction toward the bottom surface. Controller 40 may keep outlet 22a at printing start position Ps for a prescribed period after outlet 22a reached printing start position Ps, and may start application of the pressure in direction of discharge D1 while the outlet stays.

### [Control Method]

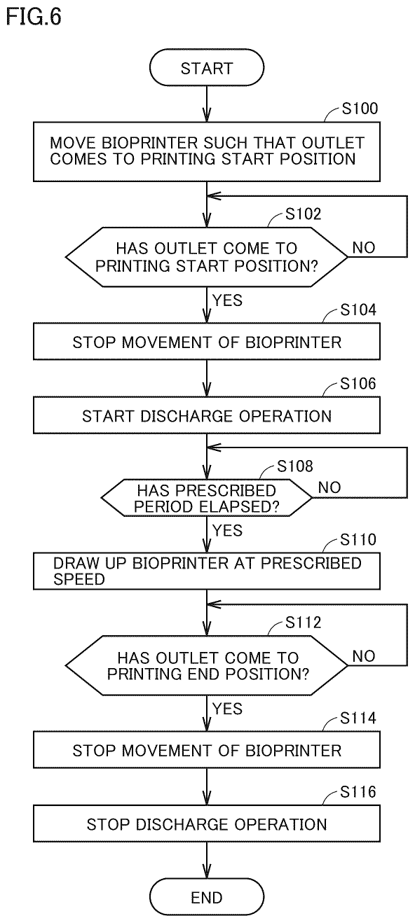

FIG. 6 is a flowchart showing a process to be performed by the controller. The process shown in FIG. 6 will be described as being performed after bioink is accommodated in nozzle 224.

In S100, controller 40 controls driver 30 to move bioprinter 20 such that outlet 22a comes to printing start position Ps.

In S102, controller 40 determines whether or not outlet 22a has come to printing start position Ps. For example, driver 30 transmits an amount of drive in a horizontal direction and an upward-downward direction to controller 40. Controller 40 determines whether or not outlet 22a has come to printing start position Ps based on information received from driver 30.

When controller 40 determines that outlet 22a has come to printing start position Ps (YES in S102), in S104, it instructs driver 30 to stop movement of bioprinter 20. Controller 40 may determine that outlet 22a has come to printing start position Ps by notifying driver 30 of the amount of drive and receiving from driver 30, a notification of completion of movement by the amount of drive indicated in the notification.

In S106, controller 40 controls bioprinter 20 to start a discharge operation. Specifically, controller 40 instructs bioprinter 20 to start application of the pressure to bioink in direction of discharge D1. Upon receiving the instruction, bioprinter 20 starts movement of piston 244 in direction of discharge D1.

In S108, controller 40 determines whether or not a prescribed period has elapsed since start of the discharge operation. Prescribed time is set as appropriate by a person skilled in the art in accordance with capability of pump 24, a hardness of the substrate, and the inner diameter of nozzle 224. By way of example, in an example where capability of the pump is 2 μL/s, gelatin is adopted as the substrate, and nozzle 224 has the inner diameter of 1.6 mm, 2 s is set as the prescribed period.

When controller 40 determines that the prescribed period has elapsed since start of the discharge operation (YES in S108), in S110, it controls driver 30 to move bioprinter 20 in direction of draw-up D3 at a prescribed speed. In S106, bioprinter 20 has been instructed to start the discharge operation. Therefore, while bioprinter 20 is drawn up, the pressure is kept applied to bioink in direction of discharge D1 and bioink is discharged from outlet 22a.

In S112, controller 40 determines whether or not outlet 22a has come to a printing end position. The printing end position is, for example, in second supporter 2. For example, controller 40 determines whether or not outlet 22a has come to the printing end position based on information on the amount of drive received from driver 30.

When controller 40 determines that outlet 22a has come to the printing end position (YES in S112), in S114, it instructs driver 30 to stop movement of bioprinter 20, and in S116, it instructs bioprinter 20 to stop the discharge operation. The process then ends.

After the process ends, controller 40 may perform processing for suction of bioink into tube 22 in order to start a next printing operation. Thereafter, the controller may start again the process shown in FIG. 6.

As set forth above, culture apparatus 100 according to the present embodiment can reliably start printing from printing start position Ps by starting a draw-up operation after the pressure is applied for a prescribed period while outlet 22a stays at printing start position Ps. If application of the pressure is started while outlet 22a is moving in housing 10 in the direction toward the bottom surface, printing may be started before outlet 22a reaches printing start position Ps and an amount of consumption of bioink may increase. Culture apparatus 100 according to the present embodiment, on the other hand, starts application of the pressure after outlet 22a is located at printing start position Ps, and hence waste of bioink can be prevented.

Though bioprinter 20 includes a single tube 22 (nozzle 224) in the embodiment, a multi-nozzle dispenser including a plurality of tubes 22 (nozzles 224) may be applicable.

### [Aspects]

The embodiment described above is understood by a person skilled in the art as specific examples of aspects below.

(Clause 1) A culture apparatus according to one aspect is a culture apparatus for bioprinting for linearly printing bioink containing cells to obtain a linear cell tissue. The culture apparatus includes a bioprinter that discharges the bioink and linearly prints the bioink, a housing configured to accommodate a substrate that supports the bioink, a driver that moves the bioprinter and the housing relatively to each other, and a controller. The bioprinter includes a tube where the bioink is accommodated and a pump configured to apply a pressure to the bioink in a direction toward an outlet of the tube. The controller is configured to control the driver to locate the outlet at a printing start position within the housing accommodating the substrate and thereafter to move the outlet in a first direction in the substrate within the housing. The controller is configured to control the pump to start application of a pressure to the bioink in a direction toward the outlet after the outlet is inserted in the substrate and before the outlet starts moving from the printing start position in the first direction.

According to the culture apparatus described in Clause 1, before start of movement of the outlet from the printing start position in the first direction, application of the pressure to bioink in the direction toward the outlet is started so that bioink can be printed by a desired length.

(Clause 2) In the culture apparatus described in Clause 1, the controller is configured to control the driver to locate the outlet at the printing start position and thereafter to keep the outlet at the printing start position. The controller is configured to control the pump to start application of the pressure to the bioink in the direction toward the outlet after the outlet is located at the printing start position and before the outlet starts moving in the first direction. The controller is configured to control the driver to move the outlet in the first direction after lapse of a prescribed period since the pump started application of the pressure to the bioink in the direction toward the outlet.

According to the culture apparatus described in Clause 2, printing from the printing start position can reliably be started by applying the pressure for a prescribed period while the outlet stays at the printing start position and thereafter starting movement in the first direction. Since the culture apparatus starts application of the pressure after the outlet is located at the printing start position, waste of bioink can be prevented.

(Clause 3) In the culture apparatus described in Clause 1 or 2, the substrate includes a first supporter that supports one end of the printed linear bioink, a second supporter that supports the other end of the linear bioink, and a holding material arranged between the first supporter and the second supporter. The first supporter, the holding material, and the second supporter are accommodated in the housing in this order from a bottom surface side of the housing. The printing start position is in the first supporter. The first direction is a direction from the first supporter toward the second supporter.

According to the culture apparatus described in Clause 3, since printing is started from within the first supporter, one end of printed linear bioink can be supported by the first supporter.

(Clause 4) In the culture apparatus described in any one of Clauses 1 to 3, the tube is attachable to and removable from the pump. The pump includes a pump housing provided with a connection port to the tube and a piston configured to make reciprocating motion within the pump housing. The pump housing is configured such that a gap of a prescribed volume is provided between an opening end of the connection port and the piston when the piston moves to a position closest to the connection port in reciprocating motion.

According to the culture apparatus described in Clause 4, since the tube is attachable to and removable from the pump, it is readily replaceable. Therefore, the tube is disposable and the pump is reusable, which is hygienic and economical. Since a gap is provided in the pump housing, entry of bioink in the pump housing at the time of suction of bioink can be prevented. Therefore, in repeated use of the bioprinter, time and efforts for cleaning and sterilization of the inside of the pump can be saved.

(Clause 5) A printing method according to one aspect is a method of printing bioink in a substrate that supports the bioink, the bioink containing cells to be a cell tissue by being cultured. The printing method includes locating an outlet of a bioprinter at a printing start position in the substrate, the bioprinter being configured to linearly print the bioink, moving within the substrate, the outlet from the printing start position in a first direction, and starting application of a pressure to the bioink in the bioprinter in a direction toward the outlet after the outlet is inserted in the substrate and before the outlet starts moving in the first direction.

According to the culture apparatus described in Clause 5, before start of movement of the outlet from the printing start position in the first direction, application of the pressure to bioink in the direction toward the outlet is started so that bioink can be printed by a desired length.

(Clause 6) A program according to one aspect is a program causing a computer to perform the printing method described in Clause 5.

(Clause 7) A recording medium according to one aspect is a computer readable recording medium having the program described in Clause 6 recorded thereon.

Each embodiment disclosed herein is also intended to be carried out in combination as appropriate within the technically consistent scope. It should be understood that the embodiment disclosed herein is illustrative and non-restrictive in every respect. The scope of the present invention is defined by the terms of the patent claims rather than the description of the embodiment above and is intended to include any modifications within the scope and meaning equivalent to the terms of the patent claims.

Though an embodiment of the present invention has been described, it should be understood that the embodiment disclosed herein is illustrative and non-restrictive in every respect. The scope of the present invention is defined by the terms of the claims and is intended to include any modifications within the scope and meaning equivalent to the terms of the claims.

What is claimed is:

1. A culture apparatus for bioprinting for linearly printing bioink containing cells to obtain a linear cell tissue, the culture apparatus comprising:
a bioprinter that discharges the bioink and linearly prints the bioink;
a housing configured to accommodate a substrate that supports the bioink;
a driver that moves the bioprinter and the housing relatively to each other; and
a controller, wherein
the bioprinter includes
a tube where the bioink is accommodated, and
a pump configured to apply a pressure to the bioink in a direction toward an outlet of the tube, and
the controller is configured to
control the driver to locate the outlet at a printing start position within the housing accommodating the substrate and thereafter to move the outlet in a first direction in the substrate within the housing, and
control the pump to start application of a pressure to the bioink in a direction toward the outlet after the outlet is inserted in the substrate and before the outlet starts moving from the printing start position in the first direction.

2. The culture apparatus according to claim 1, wherein
the controller is configured to
control the driver to locate the outlet at the printing start position and thereafter to keep the outlet at the printing start position,
control the pump to start application of the pressure to the bioink in the direction toward the outlet after the outlet is located at the printing start position and before the outlet starts moving in the first direction, and
control the driver to move the outlet in the first direction after lapse of a prescribed period since the pump started application of the pressure to the bioink in the direction toward the outlet.

3. The culture apparatus according to claim 1, wherein
the substrate includes
a first supporter that supports one end of the printed linear bioink,
a second supporter that supports the other end of the linear bioink, and
a holding material arranged between the first supporter and the second supporter,
the first supporter, the holding material, and the second supporter are accommodated in the housing in this order from a bottom surface side of the housing,
the printing start position is in the first supporter, and
the first direction is a direction from the first supporter toward the second supporter.

4. The culture apparatus according to claim 1, wherein
the tube is attachable to and removable from the pump,
the pump includes
a pump housing provided with a connection port to the tube, and
a piston configured to make reciprocating motion within the pump housing, and
the pump housing is configured such that a gap of a prescribed volume is provided between an opening end of the connection port and the piston when the piston moves to a position closest to the connection port in reciprocating motion.

5. A method of printing bioink in a substrate that supports the bioink, the bioink containing cells to be a cell tissue by being cultured, the method comprising:
locating an outlet of a bioprinter at a printing start position in the substrate, the bioprinter being configured to linearly print the bioink;
moving within the substrate, the outlet from the printing start position in a first direction; and
starting application of a pressure to the bioink in the bioprinter in a direction toward the outlet after the outlet is inserted in the substrate and before the outlet starts moving in the first direction.
